(19) United States
(12) Patent Application Publication
(10) Pub. No.: US 2011/0135763 A1
(43) Pub. Date: Jun. 9, 2011

(54) Antiseptic formulation especially for prevention of complications associated with skin burns

(76) Inventor: Alexander Fish, Flushing, NY (US)

(21) Appl. No.: 12/592,820

(22) Filed: Dec. 3, 2009

(51) Int. Cl. A61K 36/53; A61P 43/00

(52) U.S. Cl. 424/745

(57) ABSTRACT

A composition of substances generally comprises effective amounts of a mixture of a DMSO solution in a concentration ranging between 50%-100%, Oregano oil, Lavender oil, and Bay Leaf oil. Preferably, the effective amounts constitute −50% for DMSO, −20% for Oregano oil, −10% for Lavender oil, and −20% for Bay Leaf oil of the total weight of composition. The preferable concentration of DMSO solution is 100%. The DMSO solution constricts small capillaries and the dilating of medium size blood vessels. The composition is preferably usable for prevention of complications associated with second-degree thermal or chemical skin bums, for stopping the bleeding, for healing insect bites or Jelly fish stings, and for healing skin cuts and abrasions. A method for preparation of the composition comprises—mixing the effective amounts of Oregano oil, Lavender oil, and Bay Leaf oil, obtaining a mixture and—adding the effective amount of DMSO solution into the mixture.

### FIELD OF THE INVENTION

The present invention primarily relates to compositions and methods for the treatment of traumatic conditions of the skin of humans or animals, including prevention of complications developed after second-degree bums such as formation of blister, infection, and scar associated therewith.

### BACKGROUND OF THE INVENTION AND THE RELATED ART

There are known a variety of compositions of substances for treatment of the skin. Exemplarily, U.S. Pat. No. 7,476,379 describes “Emu-based formulations for the treatment of damaged skin by inhibiting microbial and fungal activity” as follows: “A formula for use in treating skin aliments includes from about 20 to about 75 wt % emu oil; from about 0.10 to 33 wt % benzyl alcohol; from about 0.10 to about 33 wt % benzoin derivative; from about 0.2 to about 2 wt % allantoin; from about 0.25 to about 1.25 wt % methylparaben; from about 1.0 to about 13 wt % alkyl esters; and from about 0.01 to about 0.30 wt % propylparaben. The formula can be applied to skin of animals or humans with skin ailments. The formula can be applied is various manners, such as through an enhanced oil, a spray, an elastomeric device, a wipe, a lotion, an ointment, a cream, a gel or combinations thereof.”

Particularly for treatment of the skin subjected to bums, U.S. Pat. No. 7,390,507 teaches: “The present invention relates to compositions and methods for the treatment of traumatic conditions of the skin including radiation dermatitis, thermal bum, sunburn, dermatomyofibromas, and exposure-induced wrinkles, comprising omega-3 fish oils, tocopherols, lavender oil and a suitable amount of a pharmaceutically acceptable carrier, and optionally, one or more of the following: Sodium PCA, or MSM.” The composition thus includes lavender oil.

U.S. Pat. No. 7,141,252 teaches: “A topical composition and method of preparation is disclosed wherein olive oil, bees wax, lemon juice and boric acid is combined to yield a cream for application to burns. The composition makes the patient more comfortable and has been shown to promote more rapid healing with less scarring than many other products.”

U.S. Pat. No. 7,220,427 teaches: “a transdermal delivery system that can deliver high molecular weight pharmaceuticals and cosmetic agents to skin cells. A novel transdermal delivery system with therapeutic and cosmetic application and methods of use of the foregoing is disclosed.” The composition is therefore used for treatment of the skin.

U.S. Pat. No. 7,029,711 teaches: “A transcutaneous pain relief composition for applying to the skin, comprising a mixture of dimethyl sulfoxide, stearic acid, emulsifying wax, glycerine, emu oil, sal butter, glucosamine, coconut oil, white beeswax, active calendula oil, slippery elm oil, Chamomile oil, arnica Oil, valenrian oil, peppermint oil, grapefruit seed extract, lavender oil and distilled water is disclosed.” The pain relief composition thus includes both: dimethyl sulfoxide (“the effective range of said dimethyl sulfoxide is between 1% and 16% by weight” of the final composition, wherein DMSO itself is used in unspecified concentration) and lavender oil (“the effective range of said lavender oil is between 0.05% and 3.5% by weight” of the final composition).

U.S. Pat. No. 7,419,689 teaches: “plant based formulations for improving liver health by protecting the liver from alcohol and chemical insults and/or by inducing phase II enzymes. Formulations according to the present invention include wasabi root fiber powder, artichoke leaf extract, asparagus dehydrate, kudzu root extract, oregano extract, schisandra berry extract, notoginseng (ethanol extract of Panax notoginseng root), sanchi (water extracts from Panax notoginseng root), Gegen root extract (Pueraria omeiensis), spinach dehydrate, or combinations thereof.” It further describes “For the ethanol protection and CCl.sub.4 assessments, stock sample solutions are made in DMSO, then diluted in cell culture media for testing. Treatment of HepG2 cells (human liver cell line) is done by adding 100 .mu.L of sample to each of three wells of a 96-well microtiter plate.” Though it's used for ‘protecting the liver from alcohol and chemical insults’, not for treatment of the skin, the composition includes oregano extract. Thus, according to U.S. Pat. No. 7,419,689, DMSO in unspecified concentration was used ‘for . . . CCl.sub.4 assessments’, but was not included in the formulations.

U.S. Pat. No. 7,470,440 teaches: “The inventive subject matter relates to methods for treating prostate cancer, comprising administration of a composition comprising therapeutically effective amounts of supercritical extracts of rosemary, turmeric, oregano and ginger; and therapeutically effective amounts of hydroalcoholic extracts of holy basil, ginger, turmeric, Scutellaria baicalensis, rosemary, green tea, huzhang, Chinese goldthread, and barberry.” Additionally, “A series of dilutions of the inventive composition in DMSO were prepared, and the dilutions were added to LNCaP growth medium so that all doses tested had equivalent (0.1%) DMSO levels. Cell growth curves were prepared by counting cells at 24, 48, and 72 hours, and were compared to control cells treated at the same times with 0.1% DMSO alone. Apoptosis in these cultures was evaluated by Western blot analysis of PARP cleavage and measurement of caspase-3 activity using a calorimetric substrate assay. Effects on purified COX-2 enzyme activity was measured using a calorimetric assay, and effects on COX-2 protein expression was determined via Western blot analysis of protein extracts from treated cells. Activities were also compared to the effects of purified curcumin dissolved in DMSO, at levels equivalent to those that would be found in the inventive compositions at similar doses.” The composition is used ‘for treating prostate cancer’. It includes oregano extract. For testing purposes, the composition was diluted in 0.1% DMSO.

### OBJECTS AND BRIEF DESCRIPTION OF THE INVENTION

The primary aim of the instant invention is to provide a composition (formulation) preferably usable for curing burns, though other applications of the composition, as described herein below, might be employed as well. More specifically, the composition should be capable of constricting small capillaries and the dilating of medium size blood vessels that contributes to preventing the formation of blister, infection, and scaring, typically associated with second-degree burns. It is desirable to provide a composition capable of stopping the bleeding. It is desirable to provide a composition that would have anti-bacterial and anti-fungal properties. It is desirable to provide a composition capable of healing insect and similar bites. It is also desirable to provide a composition that would substantially consist of all-natural ingredients.

Other aims of the invention can become apparent to those skilled in the art upon learning the present disclosure.

Second-degree burns are known to manifest as erythema with superficial blistering of the skin, and can involve more or less pain depending on the level of nerve involvement. Second-degree burns involve the superficial (papillary) dermis and may also involve the deep (reticular) dermis layer. Analyzing the related art patents enumerated above in light of the aforementioned aims of the instant invention, one can notice that the foregoing related art patents do not specifically teach healing compositions and/or methods for preventing the formation of blister, infection, and scaring associated with second-degree burns.

During a series of experiments, the instant inventor has proven that DMSO in a concentration ranging between 50% and 100% constricts small capillaries and the dilating of medium size blood vessels. The higher the DMSO concentration was taken, the better result was achieved. Such phenomenon may effectively contribute to stopping leakage of capillaries and formation of blister, whereas the leakage and blister are the major factors leading to infections and delayed wound healing, and therefore to scar formation. In the other words, if capillaries, affected by DMSO, are constricted, the lymphatic fluid does not leave the blood vessel, therefore a distention of thinned epidermis does not occur, which causes the anti-blistering effect. The best results have been observed for usage of DMSO in the concentration of substantially 100%.

During the experiments, the instant inventor has also observed that this property of DMSO has been amplified in combinations with antiseptic properties of Oregano, Lavender, and Bay Leaf essential oils (all of them are strong natural antiseptics that allow diminishing proliferation and bacterial and fungal overgrowth), and with muscle relaxant properties of Lavender oil and Bay Leaf oil (commonly known as a food additive). It should be noted that each of the aforesaid essential oils has its own anti-bacterial and anti-fungal spectrum. Lavender oil additionally balances specific smelt (odor) produced by the other ingredients. The experiments have shown that combinations of these four ingredients in predetermined effective amounts allow achieving the aforementioned aims.

### EXEMPLARY PREFERRED EMBODIMENTS OF THE INVENTION

While the invention may be susceptible to embodiment in different forms, there will be described in detail herein, specific exemplary embodiments of the present invention, with the understanding that the present disclosure is to be considered an exemplification of the principles of the invention, and is not intended to limit the invention to that as illustrated and described herein.

### Ingredients

(a) Lavender oil is known as colorless to yellow or green-yellow essential oil with sweet aroma and bitter taste; distilled from fresh flowers of several species of lavender (Lavandula); main components are linalool, linalyl acetate, geraniol, cumarin, furfurol, and borneol; used in perfumery, and in medicine as a stimulant.

Lavender is carminative, antiseptic, and soothing. It is known amongst herbalists as a holistic relaxant or as “the balancing” herb. Lavender extracts have been analyzed and the major components, linalool and linalyl acetate, are just two among of the hundreds of different active compounds identified. Other significant components include triterpenes (e.g. ursolic), flavonoids (e.g. luteolin) and coumarins (A. Y. Leung et al., Encyclopedia of Common Ingredients Used in Foods and Cosmetics, 1996, pp 339-342). The ursolic acid and other components are known to be anti-inflammatory. The cosmeceutical benefits claimed for ursolic acid include restoration of overall health and functionality of photoaged skin, in addition to facilitation of tissue repair. Ursolic acid is a potent elastase inhibitor (Q. Ying et al., “Inhibition of Human Leukocyte Elastase by Ursolic Acid; Evidence for a Hydrophobic Binding Site for Pentacyclic Triterpenes, 1991, Biochem. J. 277, 521-526). Skin is a very dynamic tissue with degradation taking place alongside repair and renewal. Compositions containing lavender extract improve skin integrity by slowing elastin degradation with respect to natural ongoing synthesis.

The mechanism by which lavender extracts ‘relax’ and reduce muscle tension has been studied in depth. M. Lis-Balchin et al., “Studies on the Mode of Action of the Essential Oil of Lavender (Lavender Angustifolia)”, Phytother. Res., 1999, 13(6), 540-542), showed that muscle tension is reduced through a postsynaptic action and not via an atropine-like mechanism and that the action of linalool reflected that of the whole lavender oil. (H. M. Kim et al., “Lavender Oil Inhibits Immediate Type Allergic Reaction in Mice and Rats,” J Pharm Pharmacol, 1999, 51(2), 221-226, were more interested in lavender's ‘soothing’ properties. They studied the effects of lavender oil on mast cell-mediated allergic reactions in mice and rats and showed that the components of lavender oil inhibit immediate-type allergic reactions by inhibition of mast cell degranulation in vivo and in vitro. Other researchers have shown that when lavender is applied topically it stimulates the local circulation (Chevallier, 1996).

(b) Oregano oil is a name for several herbs used for flavoring food. A plant of the family Labiatae (mint family), Origanum vulgare, also called Spanish thyme and wild marjoram, is the usual source for the spice sold as oregano in the Mediterranean countries and in the United States. Its flavor is similar to that of marjoram of sweet marjoram, but slightly less sweet. In Spain and Italy many other Origanum species are also grown as oregano. A related herb (Coleus amboinicius) of the same family, called suganda in its native Indomalaysia, is known as oregano in the Philippines and Mexico, where it is a popular flavoring. Several other herbs also provide spices called oregano, e.g., species of Lippia and Lantana of the verbena family. In all cases the flavoring is made from the dried herbage. Oregano is classified in the division Magnoliophyta, class Magnoliopsida, order Lamiales, family Labiatae.

Numerous university studies have shown that Oil of Oregano is a highly potent purifier that provides many benefits for human health. Oil of Oregano is a natural substance that is extracted from wild oregano plants, and two key compounds found in it are carvacrol and thymol. Studies have shown that both of these compounds have significant effects on harmful microorganisms that cause many illnesses in humans. It is important not to confuse Oil of Oregano with common oregano that is used as a spice for cooking. Common oregano is typically Origanum Marjoram, while Oil of Oregano is derived from Origanum Vulgare.

Oil of Oregano can be purchased as either a liquid or as capsules/tablets. In both forms, it is important to verify that the oil is derived from the proper potent oregano plant, and that the carvacrol concentration is at least 70%.

The ancient Greeks were one of the first people to recognize oregano oil for its health benefits and medicinal qualities. It is known to be a potent antiviral, antibacterial, antifungal, and antiparasitic oil that can reduce pain and inflammation, and effectively fight off infections.

Some of the specific benefits of Oil of Oregano are:—destroying organisms that contribute to skin infections and digestive problems;—strengthening the immune system;—increasing joint and muscle flexibility;—improving respiratory health.

(c) Bay Leaf oil is the dried aromatic leaf of the laurel or bay (Laurus nobilis) commonly used as a seasoning in cooking, food or color additives. Bay Leaf oil also exhibits superior muscle relaxant, anti-fungal, and antiseptic properties.

(d) DMSO (dimethyl sulfoxide) is known as an industrial solvent that has the ability to penetrate plant and animal tissues and to preserve living cells during freezing. It has bacteriostatic, anti-inflammatory, analgesic, radioprotective, fibrinolytic and vasodilative properties, and inhibits cholinesterase and granulation tissue. DMSO also has been used as an agent to increase the penetrability of other substances. As mentioned above, the instant inventor has proven that DMSO in a concentration ranging between 50% and 100% constricts small capillaries and also constricts the dilating of medium size blood vessels. This capability of DMSO has increased with an increase of the DMSO concentration, and the best results have been observed for usage of DMSO (USP grade) in the concentration essentially of 100%.

Accordingly, the inventive composition generally comprises effective amounts of a mixture of: (a) a DMSO solution in a concentration ranging between 50%-100%, (b) Oregano oil, (c) Lavender oil, and (d) Bay Leaf oil.

In a preferred embodiment, the inventive composition comprises a mixture of: (a) a DMSO solution in a concentration ranging between 50%-100% in an amount of 50% by weight, (b) Oregano oil in an amount of 20% by weight, (c) Lavender oil in an amount of 10% by weight, and (d) Bay Leaf oil in an amount of 20% by weight.

In another preferred embodiment, the inventive composition comprises a mixture of: (a) a DMSO solution in a concentration of 100% in an amount of 50% by weight, (b) Oregano oil in an amount of 20% by weight, (c) Lavender oil in an amount of 10% by weight, and (d) Bay Leaf oil in an amount of 20% by weight.

According to the invention, a method for preparation of the inventive composition comprises the steps of: mixing effective amounts of Oregano oil, Lavender oil, and Bay Leaf oil; and adding into the mixture a DMSO solution having a concentration in a range of between 50% and 100%.

In another preferred embodiment, a method for preparation of the inventive composition comprises the steps of: mixing Oregano oil in an amount of 20% by weight, Lavender oil in an amount of 10% by weight, and Bay Leaf oil in an amount of 20% by weight; and adding into the mixture a DMSO solution having a concentration in a range of between 50% and 100% in an amount of 50% by weight.

In another preferred embodiment, a method for preparation of the inventive composition comprises the steps of: mixing Oregano oil in an amount of 20% by weight, Lavender oil in an amount of 10% by weight, and Bay Leaf oil in an amount of 20% by weight; and adding into the mixture a DMSO solution having a concentration of 100% in an amount of 50% by weight.

According to the invention, a method for using the inventive composition in any of the above-described embodiments by a patient, having a damaged skin area, comprises the steps of: shaking content of the inventive composition, and applying the inventive composition directly to the damaged skin area.

### EXAMPLES OF USAGE OF THE INVENTIVE COMPOSITION

Experimental clinical observations include collecting data from approximately 25 patients, who have experienced burns. Below are some examples of the observations.

### Example 1

A patient was barbequing, and misstepped by touching the burning grill with the entire palm. His mother applied the inventive composition immediately. The patient had intense burning sensation for the next 5 to 10 minutes. After one hour, 30 people who participated at the event could not even see redness on his palm.

### Example 2

The inventive composition was experimentally used in private practice of the instant inventor. A phenol and alcohol permanent distraction of the nail root and matrix (also known as an P&A procedure) is widely known in podiatry. The P&A procedure effectively is a chemical burn of the root of the nail. A conventional method prescribes using a 1% Silvadene cream after such procedure. A significant diminishing of drainage from the wound, inflammatory reaction, pain and discomfort, and recovery time after the surgery have been observed, where the inventive composition has been used for healing the skin damaged with the chemical burn. The instant inventor has used the inventive composition directly applying it to the burn after the surgery. It has been noted that post-operative pain was diminished to none, and post-operative drainage and edema were also significantly diminished. The most important observation was that morbidity and the healing period were about twice shorter than those for the conventional method of healing.

### Additional Exemplary Applications of the Inventive Composition

According to the instant inventor's experiments, the inventive formulation could also be employed to stop bleeding, even on patients who take blood thinners, e.g. Coumadin. A person with intensive bleeding was going to a hospital, but after the treatment by the inventive composition, the bleeding was stopped in 2 minutes.

The inventive composition can be used for healing insect (e.g. mosquito, bees, etc.) bites and Jelly fish stings. As an example, the inventor used it 5 times and succeeded in instant neutralization of complications associated with Jelly fish stings.

The inventive composition can be utilized for healing skin cuts and abrasions, significantly diminishing the healing time. For instance, a seven year old girl cut her face. The mother immediately used the composition, and the face was healed without a scar.

The inventive composition can be employed for treatment of fungal infections of nails. At least 20 patients with superficial fungal infections of nails have been experimentally cured completely.

I claim:

1. A composition of substances comprising effective amounts of a mixture of: (a) a DMSO solution in a concentration ranging between 50% and 100%, (b) Oregano oil, (c) Lavender oil, and (d) Bay Leaf oil.

2. The composition according claim 1, wherein said DMSO solution is provided in a concentration of substantially 100%.

3. The composition according claim 1, wherein the effective amount of said DMSO solution constitutes 50% of the total weight of said composition; the effective amount of said Oregano oil constitutes 20% of the total weight of said composition; the effective amount of said Lavender oil constitutes 10% of the total weight of said composition; and the effective amount of said Bay Leaf oil constitutes 20% of the total weight of said composition.

4. The composition according claim 3, wherein said DMSO solution is provided in a concentration of substantially 100%.

5. The method for preparation of the composition according to claim 1, said method comprises the steps of: mixing said effective amounts of Oregano oil, Lavender oil, and Bay Leaf oil, obtaining a mixture; and adding said DMSO solution into the mixture.

6. The method for preparation of the composition according to claim 2, said method comprises the steps of: mixing said effective amounts of Oregano oil, Lavender oil, and Bay Leaf oil, obtaining a mixture; and adding said DMSO solution into the mixture.

7. The method for preparation of the composition according to claim 3, said method comprises the steps of: mixing said effective amounts of Oregano oil, Lavender oil, and Bay Leaf oil, obtaining a mixture; and adding said DMSO solution into the mixture.

8. The method for preparation of the composition according to claim 4, said method comprises the steps of mixing said effective amounts of Oregano oil, Lavender oil, and Bay Leaf oil, obtaining a mixture; and adding said DMSO solution into the mixture.

9. The method for using the composition of claim 1 by a person having a damaged skin area, said method comprises the steps of: shaking content of said composition, and applying said composition directly to the damaged skin area.

10. The method of claim 9, wherein said composition is used for at least one of the following purposes: for prevention of complications associated with second-degree skin burns, for stopping the bleeding, for healing insect bites or Jelly fish stings, for healing fungal infection, and for healing skin cuts and abrasions.

11. The method for using the composition of claim 2 by a person having a damaged skin area, said method comprises the steps of: shaking content of said composition, and applying said composition directly to the damaged skin area.

12. The method of claim 11, wherein said composition is used for at least one of the following purposes: for prevention of complications associated with second-degree skin burns, for stopping the bleeding, for healing insect bites or Jelly fish stings, for healing fungal infection, and for healing skin cuts and abrasions.

13. The method for using the composition of claim 3 by a person having a damaged skin area, said method comprises the steps of: shaking content of said composition, and applying said composition directly to the damaged skin area.

14. The method of claim 13, wherein said composition is used for at least one of the following purposes: for prevention of complications associated with second-degree skin bums, for stopping the bleeding, for healing insect bites or Jelly fish stings, for healing fungal infection, and for healing skin cuts and abrasions.

15. The method for using the composition of claim 4 by a person having a damaged skin area, said method comprises the steps of: shaking content of said composition, and applying said composition directly to the damaged skin area.

16. The method of claim 15, wherein said composition is used for at least one of the following purposes: for prevention of complications associated with second-degree skin burns, for stopping the bleeding, for healing insect bites or Jelly fish stings, for healing fungal infection, and for healing skin cuts and abrasions.
